(19) United States
(12) Patent Application Publication
(10) Pub. No.: US 2021/0082539 A1
(43) Pub. Date: Mar. 18, 2021

(54) GENE MUTATION IDENTIFICATION METHOD AND APPARATUS, AND STORAGE MEDIUM

(71) Applicant: BEIJING SENSETIME TECHNOLOGY DEVELOPMENT CO., LTD., Beijing (CN)

(72) Inventor: Zhiqiang HU, Beijing (CN)

(73) Assignee: BEIJING SENSETIME TECHNOLOGY DEVELOPMENT CO., LTD., Beijing (CN)

(21) Appl. No.: 17/102,136

(22) Filed: Nov. 23, 2020

Related U.S. Application Data
(63) Continuation of application No. PCT/CN2019/089499, filed on May 31, 2019.

(30) Foreign Application Priority Data
Mar. 29, 2019 (CN) 201910251891.0

(51) Int. Cl. G16B 30/10; G16B 20/20; G06N 3/12

(52) U.S. Cl. CPC G16B 30/10; G06N 3/126; G16B 20/20

(57) ABSTRACT

The present disclosure relates to a gene mutation identification method and apparatus, and a storage medium. The method includes: obtaining at least one gene sequencing read segment corresponding to a gene mutation candidate site; determining a sequence feature and a non-sequence feature of the gene mutation candidate site according to attribute information of the at least one gene sequencing read segment, where the sequence feature is a feature related to the position of the site; and identifying gene mutation of the gene mutation candidate site based on the sequence feature and the non-sequence feature. According to embodiments of the present disclosure, the sequence feature and the non-sequence feature of the gene can be combined, thereby more comprehensively analyzing the features of a gene mutation site and improving accuracy of gene mutation identification.

### CROSS-REFERENCE TO RELATED APPLICATIONS

The present disclosure is a bypass continuation of and claims priority under 35 U.S.C. § 111(a) to PCT Application. No. PCT/CN2019/089499, filed on May 31, 2019, which claims priority to Chinese Patent Application No. 201910251891.0, filed to the Chinese Patent Office on Mar. 29, 2019, and entitled “GENE MUTATION IDENTIFICATION METHOD AND APPARATUS, AND STORAGE MEDIUM”, each of which is incorporated herein by reference in its entirety.

### TECHNICAL FIELD

The present disclosure relates to the field of computer technologies, and in particular, to a gene mutation identification method and apparatus, and a storage medium.

### BACKGROUND

With the development of biotechnology, a human gene sequence may be determined by a gene sequencing technology, and gene sequence analysis may serve as a basis for further gene research and modification. At present, compared with the first-generation gene sequencing technology, the second-generation gene sequencing technology greatly improves the efficiency of gene sequencing, reduces the costs of gene sequencing, and maintains the accuracy of gene sequencing. If the first-generation sequencing technology may take three years to complete sequencing of a human genome, then the time may be shortened to one week by using the second-generation sequencing technology.

### SUMMARY

In this regard, the present disclosure provides a gene mutation identification solution.

A gene mutation identification method provided according to one aspect of the present disclosure includes:
obtaining at least one gene sequencing read segment corresponding to a gene mutation candidate site;
determining a sequence feature and a non-sequence feature of the gene mutation candidate site according to attribute information of the at least one gene sequencing read segment, where the sequence feature is a feature related to the position of the site; and
identifying gene mutation of the gene mutation candidate site based on the sequence feature and the non-sequence feature.

In one possible implementation, the attribute information includes sequence attribute information; and determining the sequence feature of the gene mutation candidate site according to the attribute information of the at least one gene sequencing read segment includes:
determining a preset site interval where the gene mutation candidate site is located according to gene position information of the gene mutation candidate site;
obtaining the sequence attribute information of the at least one gene sequencing read segment at each site in the preset site interval, where the sequence attribute information is information representing a gene attribute and related to the position of the site; and
generating the sequence feature of the gene mutation candidate site according to the sequence attribute information at each site in the preset site interval.

In one possible implementation, obtaining the sequence attribute information of the at least one gene sequencing read segment at each site in the preset site interval includes:
determining a gene type of the at least one gene sequencing read segment at each site; and
counting the number of genes of each gene type corresponding to each site.

In one possible implementation, obtaining the sequence attribute information of the at least one gene sequencing read segment at each site in the preset site interval includes:
determining a gene type of a deletion gene of each gene sequencing read segment at each site according to a comparison result between a gene sequence of each gene sequencing read segment and a gene sequence of a reference genome; and
counting the number of deletion genes of each gene type of the at least one gene sequencing read segment at each site.

In one possible implementation, obtaining the sequence attribute information of the at least one gene sequencing read segment at each site in the preset site interval includes:
determining a gene type of an insertion gene of each gene sequencing read segment at each site according to the comparison result between the gene sequence of each gene sequencing read segment and the gene sequence of the reference genome; and
counting the number of insertion genes of each gene type of the at least one gene sequencing read segment at each site.

In one possible implementation, the sequence attribute information includes at least one of the following:
the gene type of a reference gene; the number of genes of each gene type; the number of deletion genes of each gene type; or the number of insertion genes of each gene type.

In one possible implementation, the attribute information includes non-sequence attribute information; and determining the non-sequence feature of the gene mutation candidate site according to the attribute information of the at least one gene sequencing read segment includes:
obtaining the non-sequence attribute information of the at least one gene sequencing read segment, where the non-sequence attribute information is information representing a gene attribute and unrelated to the position of the site; and
determining the non-sequence feature of the gene mutation candidate site according to the non-sequence attribute information of the at least one gene sequencing read segment.

In one possible implementation, the non-sequence information includes at least one of the following:
comparison quality; positive and negative strand preference; gene sequencing read segment length; or edge preference.

In one possible implementation, determining the non-sequence feature of the gene mutation candidate site according to the non-sequence attribute information of the at least one gene sequencing read segment includes:
determining the comparison quality of each gene sequencing read segment according to the comparison quality of each site in each gene sequencing read segment, where the comparison quality is used for representing the accuracy of gene sequencing of each gene sequence in the gene sequencing read segment; and
determining the non-sequence feature corresponding to the gene mutation candidate site according to the comparison quality of each gene sequencing read segment.

In one possible implementation, determining the non-sequence feature of the gene mutation candidate site according to the non-sequence attribute information of the at least one gene sequencing read segment includes:
determining a positive and negative strand ratio of gene strands to which the at least one gene sequencing read segment belongs according to positive and negative strand information of a gene strand to which each gene sequencing read segment belongs; and
determining the non-sequence feature corresponding to the gene mutation candidate site according to the positive and negative strand ratio.

In one possible implementation, identifying the gene mutation of the gene mutation candidate site based on the sequence feature and the non-sequence feature includes:
performing feature integration on the sequence feature and the non-sequence feature to obtain an integrated feature of the gene mutation candidate site; and
identifying the gene mutation of the gene mutation candidate site based on the integrated feature of the gene mutation candidate site.

In one possible implementation, the identifying the gene mutation of the gene mutation candidate site based on the integrated feature of the gene mutation candidate site includes:
obtaining a mutation value of gene mutation of the gene mutation candidate site according to the integrated feature of the gene mutation candidate site; and
if the mutation value is greater than or equal to a preset threshold, determining the existence of gene mutation of the gene mutation candidate site.

In one possible implementation, obtaining at least one gene sequencing read segment corresponding to the gene mutation candidate site includes:
obtaining a gene sequencing read segment obtained by performing gene sequencing on a somatic gene;
comparing the gene sequence of the gene sequencing read segment with the gene sequence of the reference genome to obtain a comparison result;
determining the gene mutation candidate site of an abnormal gene of the somatic gene according to the comparison result; and
obtaining the at least one gene sequencing read segment corresponding to the gene mutation candidate site.

A gene mutation identification apparatus provided according to another aspect of the present disclosure includes:
an obtaining module, configured to obtain at least one gene sequencing read segment corresponding to a gene mutation candidate site;
a determination module, configured to determine a sequence feature and a non-sequence feature of the gene mutation candidate site according to attribute information of the at least one gene sequencing read segment, where the sequence feature is a feature related to the position of the site; and
an identification module, configured to identify gene mutation of the gene mutation candidate site based on the sequence feature and the non-sequence feature.

In one possible implementation, the attribute information includes sequence attribute information; and the determination module includes:
a first determination sub-module, configured to determine a preset site interval where the gene mutation candidate site is located according to gene position information of the gene mutation candidate site;
a first obtaining sub-module, configured to obtain the sequence attribute information of the at least one gene sequencing read segment at each site in the preset site interval, where the sequence attribute information is information representing a gene attribute and related to the position of the site; and
a first generation sub-module, configured to generate the sequence feature of the gene mutation candidate site according to the sequence attribute information at each site in the preset site interval.

In one possible implementation, the first obtaining sub-module is specifically configured to: determine a gene type of the at least one gene sequencing read segment at each site; and count the number of genes of each gene type corresponding to each site.

In one possible implementation, the first obtaining sub-module is specifically configured to: determine a gene type of a deletion gene of each gene sequencing read segment at each site according to a comparison result between a gene sequence of each gene sequencing read segment and a gene sequence of a reference genome; and count the number of deletion genes of each gene type of the at least one gene sequencing read segment at each site.

In one possible implementation, the first obtaining sub-module is specifically configured to: determine a gene type of an insertion gene of each gene sequencing read segment at each site according to the comparison result between the gene sequence of each gene sequencing read segment and the gene sequence of the reference genome; and count the number of insertion genes of each gene type of the at least one gene sequencing read segment at each site.

In one possible implementation, the sequence attribute information includes at least one of the following:
the gene type of a reference gene; the number of genes of each gene type; the number of deletion genes of each gene type; or the number of insertion genes of each gene type.

In one possible implementation, the attribute information includes non-sequence attribute information; and the determination module includes:
a second obtaining sub-module, configured to obtain the non-sequence attribute information of the at least one gene sequencing read segment, where the non-sequence attribute information is information representing a gene attribute and unrelated to the position of the site; and
a second determination sub-module, configured to determine the non-sequence feature of the gene mutation candidate site according to the non-sequence attribute information of the at least one gene sequencing read segment.

In one possible implementation, the non-sequence information includes at least one of the following:
comparison quality; positive and negative strand preference; gene sequencing read segment length; or edge preference.

In one possible implementation, the second determination sub-module is specifically configured to: determine the comparison quality of each gene sequencing read segment according to the comparison quality of each site in each gene sequencing read segment, where the comparison quality is used for representing the accuracy of gene sequencing of each gene sequence in the gene sequencing read segment; and determine the non-sequence feature corresponding to the gene mutation candidate site according to the comparison quality of each gene sequencing read segment.

In one possible implementation, the second determination sub-module is specifically configured to: determine a positive and negative strand ratio of gene strands to which the at least one gene sequencing read segment belongs according to positive and negative strand information of a gene strand to which each gene sequencing read segment belongs; and determine the non-sequence feature corresponding to the gene mutation candidate site according to the positive and negative strand ratio.

In one possible implementation, the identification module includes:
an integration sub-module, specifically configured to perform feature integration on the sequence feature and the non-sequence feature to obtain an integrated feature of the gene mutation candidate site; and
an identification sub-module, configured to identify the gene mutation of the gene mutation candidate site based on the integrated feature of the gene mutation candidate site.

In one possible implementation, the identification sub-module is specifically configured to: obtain a mutation value of gene mutation of the gene mutation candidate site according to the integrated feature of the gene mutation candidate site; and if the mutation value is greater than or equal to a preset threshold, determine the existence of gene mutation of the gene mutation candidate site.

In one possible implementation, the obtaining module is specifically configured to:
obtain a gene sequencing read segment obtained by performing gene sequencing on a somatic gene;
compare the gene sequence of the gene sequencing read segment with the gene sequence of the reference genome to obtain a comparison result;
determine the gene mutation candidate site of an abnormal gene of the somatic gene according to the comparison result; and
obtain at least one gene sequencing read segment corresponding to the gene mutation candidate site.

A gene mutation identification apparatus provided according to another aspect of the present disclosure includes: a processor; and a memory configured to store processor-executable instructions, where the processor is configured to execute the foregoing method.

A non-volatile computer-readable storage medium provided according to another aspect of the present disclosure has computer program instructions stored thereon, where the foregoing method is implemented when the computer program instructions are executed by a processor.

According to the embodiments of the present disclosure, at least one gene sequencing read segment corresponding to a gene mutation candidate site is obtained, and a sequence feature and a non-sequence feature of the gene mutation candidate site are determined according to attribute information of the at least one gene sequencing read segment, so that gene mutation of the gene mutation candidate site is identified based on the determined sequence feature and non-sequence feature, where the sequence feature may be a feature related to the position of the site, and the non-sequence feature may be a feature unrelated to the position of the site. Therefore, in the gene mutation identification process, the sequence feature and the non-sequence feature of the gene may be combined, thereby more comprehensively analyzing the feature of a gene mutation site, screening out germ line gene mutation and avoiding interference caused by noise and errors, better identifying gene mutation, and improving the accuracy of gene mutation identification.

Exemplary embodiments are described in detail below according to the following reference accompanying drawings, and other features and aspects of the present disclosure become clear.

### BRIEF DESCRIPTION OF THE DRAWINGS

The accompanying drawings included in the description and constructing a part of the description illustrate the exemplary embodiments, features, and aspects of the present disclosure together with the description, and are intended to explain the principles of the present disclosure.

FIG. 1 is a flowchart of a gene mutation identification method according to the embodiments of the present disclosure.

FIG. 2 is a flowchart of obtaining at least one gene sequencing read segment corresponding to a gene mutation candidate site according to the embodiments of the present disclosure.

FIG. 3 is a flowchart of a process of determining a sequence feature of a gene mutation candidate site according to the embodiments of the present disclosure.

FIG. 4 is a flowchart of a process of determining a non-sequence feature of a gene mutation candidate site according to the embodiments of the present disclosure.

FIG. 5 is a flowchart of a process of identifying gene mutation of a gene mutation candidate site according to the embodiments of the present disclosure.

FIG. 6 is a block diagram of a neural network model according to the embodiments of the present disclosure.

FIG. 7 is a block diagram of a gene mutation identification apparatus according to the embodiments of the present disclosure.

FIG. 8 is a block diagram of a gene mutation identification apparatus illustrated according to an exemplary embodiment of the present disclosure.

### DETAILED DESCRIPTION

The following describes various exemplary embodiments, features, and aspects of the present disclosure in detail with reference to the accompanying drawings. Same reference numerals in the accompanying drawings represent elements with same or similar functions. Although various aspects of the embodiments are illustrated in the accompanying drawings, the accompanying drawings are not necessarily drawn in proportion unless otherwise specified.

The special term “exemplary” here refers to “being used as an example, the embodiments, or an illustration”. Any embodiment described as “exemplary” here should not be explained as being more superior or better than other embodiments.

In addition, for better illustration of the present disclosure, various specific details are given in the following specific implementations. A person skilled in the art should understand that the present disclosure may also be implemented without the specific details. In some instances, methods, means, elements, and circuits well known to a person skilled in the art are not described in detail so as to highlight the subject matter of the present disclosure.

According to the gene mutation identification solution provided in the embodiments of the present disclosure, at least one gene sequencing read segment corresponding to a gene mutation candidate site is obtained, so that gene mutation of the gene mutation candidate site is identified according to the at least one gene sequencing read segment. In the gene mutation identification process, a sequence feature is generated according to sequence attribute information of the at least one gene sequencing read segment, a non-sequence feature is generated according to non-sequence attribute information of the at least one gene sequencing read segment, and then the gene mutation of the gene mutation candidate site is identified by means of the sequence feature and the non-sequence feature, so that the sequence attribute information and the non-sequence attribute information of the at least one gene sequencing read segment may be integrated, and the sequence attribute information of the gene sequencing read segment is more comprehensively utilized.

In the related art, gene mutation identification is generally performed by using a conventional machine learning method such as support vector machine and random forest. Although the method is simple to implement, it is difficult to utilize sequence attribute information of a gene sequence near the gene mutation candidate site, and the effect of gene mutation identification may get into a bottleneck after the gene data amount increases to a certain extent. Some related arts employ a deep learning method that identifies gene mutation by utilizing a neural network. However, it is difficult for the neural network to integrate non-sequence information of a gene sequence, and thus, gene data cannot be analyzed more comprehensively. In the embodiments of the present disclosure, in the gene mutation identification process, a sequence feature and a non-sequence feature of the gene mutation candidate site are extracted by utilizing a neural network model integrated with multi-modal information, so that sequence attribute information and non-sequence attribute information of a gene sequence may be integrated, thereby more comprehensively analyzing gene data, screening out germ line gene mutation and avoiding interference caused by noise and errors, and better identifying gene mutation. The following embodiments describe the gene mutation identification process in detail.

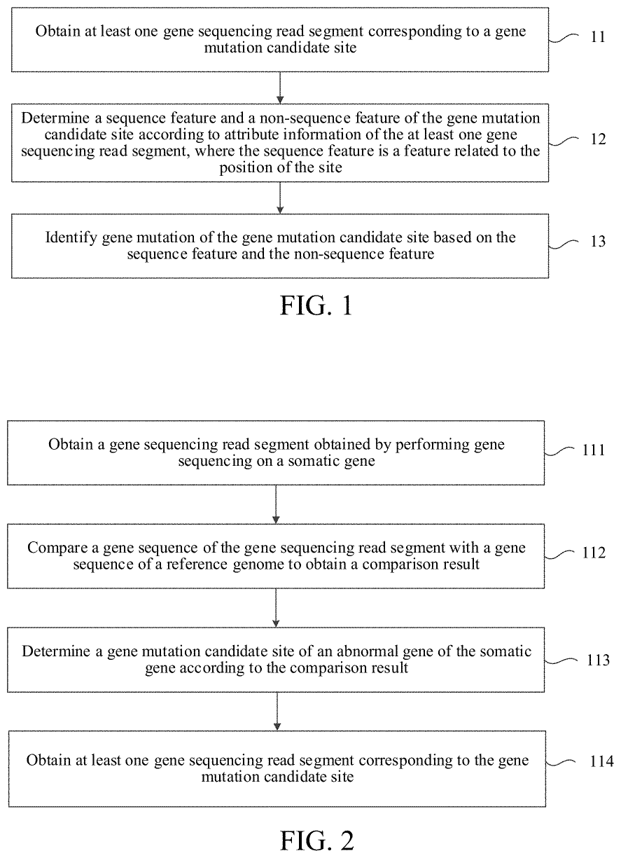

FIG. 1 is a flowchart of a gene mutation identification method according to the embodiments of the present disclosure. The gene mutation identification method may be executed by a gene mutation identification apparatus or other processing devices, where the gene mutation identification apparatus may be a User Equipment (UE), a mobile device, a user terminal, a terminal, a cellular phone, a cordless phone, a Personal Digital Assistant (PDA), a handheld device, a computing device, a vehicle-mounted device, a wearable device, or the like. Or, the gene mutation identification apparatus may be a server. In some possible implementations, the gene mutation identification method may be implemented by a processor by invoking computer readable instructions stored in a memory.

As shown in FIG. 1, the gene mutation identification method includes the following steps.

At step 11, at least one gene sequencing read segment corresponding to a gene mutation candidate site is obtained.

In the embodiments of the present disclosure, the gene mutation identification apparatus obtains a gene sequencing read segment obtained by gene sequencing, and then obtains at least one gene sequencing read segment corresponding to a gene mutation candidate site in the gene sequencing read segment obtained by gene sequencing. The gene sequencing read segment may be understood as a gene sequence labeled with a gene type after gene sequencing, and the length of each gene sequencing read segment may be the same or different. Under the condition that the length is different, the length of each gene sequencing read segment may be in a preset length range, so that the length of each gene sequencing read segment may be guaranteed to be relatively close. The gene type may be understood as a base type and may include cytosine (C), guanine (G), adenine (A), and thymine (T), so that the gene sequencing read segment may be a gene sequence including AGCT. The gene mutation candidate site herein may be a site having an abnormal gene sequence. The site of the gene sequence represents the position of the gene sequence, and at least one gene sequencing read segment may be present for each site, i.e., there may be at least one gene sequencing read segment obtained by gene sequencing at the same site. Accordingly, the gene mutation candidate site corresponds to at least one gene sequencing read segment, where the at least one gene sequencing read segment is abnormal at this site. There may be at least one gene mutation candidate site, and each gene mutation candidate site corresponds to at least one gene sequencing read segment. For ease of understanding, the embodiments of the present disclosure are described in terms of one gene variant candidate site.

At step 12, a sequence feature and a non-sequence feature of the gene mutation candidate site is determined according to attribute information of the at least one gene sequencing read segment, where the sequence feature is a feature related to the position of the site.

In the embodiments of the present disclosure, after obtaining the at least one gene sequencing read segment corresponding to the gene mutation candidate site, attribute information of the at least one gene sequencing read segment corresponding to the gene mutation candidate site may be extracted, and a sequence feature and a non-sequence feature of the gene mutation candidate site are generated according to the extracted attribute information. The attribute information includes sequence attribute information and non-sequence attribute information. The sequence attribute information is information representing a gene attribute of the gene sequencing read segment and related to the position of the site. The non-sequence attribute information is information not limited by the position of the site and representing a gene attribute. When the attribute information is extracted, a plurality of gene sequencing read segments corresponding to the gene candidate site may be randomly selected, and the attribute information of the plurality of randomly selected gene sequencing read segments is extracted; the attribute information of each gene sequencing read segment corresponding to the gene candidate site may also be extracted.

Here, when the sequence attribute information is extracted, the sequence attribute information of the at least one gene sequencing read segment at the gene mutation candidate site is extracted, and the sequence attribute information of the at least one gene sequencing read segment in the vicinity of the gene mutation candidate site is also extracted. Here, when the sequence feature of the gene mutation candidate site is determined, a neural network model with a convolutional layer and a pooling layer may be utilized, and the sequence feature of the gene mutation candidate site is extracted from the at least one gene sequencing read segment corresponding to the gene mutation candidate site. The neural network model includes two branch structures, one of which extracts the sequence feature of the gene sequencing read segment and includes the convolutional layer and the pooling layer; the other branch may extract the non-sequence feature of the gene sequencing read segment. The neural network model may integrate various modal information (sequence attribute information and non-sequence attribute information), so as to identify gene mutation of the gene mutation candidate site. When the non-sequence feature of the gene mutation candidate site is determined, the neural network model may be utilized. The non-sequence feature of the at least one gene sequencing read segment is extracted by the other branch of the neural network model. The branch structure may include a fully-connected layer that may be used to extract the non-sequence feature that is not limited by the position.

At step 13, gene mutation of the gene mutation candidate site is identified based on the sequence feature and the non-sequence feature.

In the implementations of the present disclosure, after determining the sequence feature and the non-sequence feature of the gene mutation candidate site, the sequence feature and the non-sequence feature may be fused, and the gene mutation of the gene feature candidate site is identified, for example, whether the gene of the gene mutation candidate site is mutated, or whether the gene of the gene mutation candidate site has an abnormal gene sequence due to noise and the like is determined by utilizing the neural network model.

In the embodiments of the present disclosure, gene mutation of the gene mutation candidate site is identified according to the sequence feature and the non-sequence feature of the gene mutation candidate site, so that gene sequencing data may be analyzed more comprehensively. When the gene mutation of the gene mutation candidate site is identified, at least one gene sequencing read segment corresponding to the gene mutation candidate site needs to be obtained first. The embodiments of the present disclosure also provide a process of obtaining the at least one gene sequencing read segment corresponding to the gene mutation candidate site.

FIG. 2 is a flowchart of obtaining at least one gene sequencing read segment corresponding to a gene mutation candidate site according to the embodiments of the present disclosure. In one possible implementation, obtaining the at least one gene sequencing read segment corresponding to the gene mutation candidate site includes the following steps.

At step 111, a gene sequencing read segment obtained by performing gene sequencing on a somatic gene is obtained.

Here, at least one gene sequencing read segment is obtained by performing gene sequencing on a somatic gene, and the gene sequencing read segment is a sequence labeled with a gene type of the somatic gene. After gene sequencing is performed on the somatic gene, a gene type of each gene in the gene sequencing read segment is obtained, and gene position information of the site where each gene in the gene sequencing read segment is located is also obtained. The same site may correspond to at least one gene sequencing read segment.

In one possible implementation, at least one gene sequencing read segment is obtained by performing gene sequencing on a somatic gene, and the gene sequencing read segment obtained through gene sequencing is preprocessed. The preprocessing mode includes cross contamination screening, sequencing quality screening, comparison quality screening, abnormal read segment length screening and the like. Through preprocessing, the cross-contaminated gene sequencing read segments may be screened out, and the gene sequencing read segments with low sequencing quality and comparison quality and abnormal read segment length are screened out.

At step 112, the gene sequence of the gene sequencing read segment is compared with the gene sequence of the reference genome to obtain the comparison result.

In the embodiments of the present disclosure, after obtaining a gene sequencing read segment obtained by performing gene sequencing on a somatic gene, the gene sequence of the obtained gene sequencing read segment is compared with the gene sequence of the reference genome with the same site to obtain the comparison result. For example, each gene sequencing read segment obtained through gene sequencing is compared with the gene sequence of the reference genome with the same site to determine different sites of the gene sequence of the gene sequencing read segment and the gene sequence of the reference genome. At least one gene sequencing read segment with the same site is compared with the gene sequence of the reference genome with the same site to determine different sites of the gene sequence of the at least one gene sequencing read segment and the gene sequence of the reference genome.

At step 113, the gene mutation candidate site of an abnormal gene of the somatic gene is determined according to the comparison result.

In the embodiments of the present disclosure, different sites of the gene sequences of the gene sequencing read segment and the reference genome are determined according to the comparison result. If in the at least one gene sequencing read segment corresponding to the site, the proportion of sending the mutated gene sequencing read segment at the site is greater than a preset proportion, then it can be determined that the site is a gene mutation candidate site, and otherwise, the site is not considered to be a gene mutation candidate site. The gene sequence of the gene sequencing read segment is different from that of the reference genome at the site, which is possibly caused by sequencing errors. In this way, the abnormal phenomenon of the gene sequence caused by gene sequencing errors can be avoided.

At step 114, the at least one gene sequencing read segment corresponding to the gene mutation candidate site is obtained.

In the embodiments of the present disclosure, after the gene mutation candidate site is determined, at least one gene sequencing read segment corresponding to the gene mutation candidate site is obtained. Each gene mutation candidate site corresponds to at least one gene sequencing read segment, and the gene sequence of the gene mutation candidate site can be different from the gene sequence of the reference genome with the same site. There may be at least one gene mutation candidate site.

Through the process of obtaining the at least one gene sequencing read segment corresponding to the gene mutation candidate site, the gene mutation candidate site can be accurately determined, and the at least one gene sequencing read segment corresponding to the gene mutation candidate site can also be determined in the gene sequencing read segment obtained through gene sequencing.

In the embodiments of the present disclosure, the sequence feature of the gene mutation candidate site is determined according to the sequence attribute information of the at least one gene sequencing read segment corresponding to the gene mutation candidate site, so that when the gene mutation of the gene mutation candidate site is identified, a sequence attribute of the at least one gene sequencing read segment corresponding to the gene mutation candidate site may be considered. The process of determining the sequence feature of the gene mutation candidate site is described in detail below by an example.

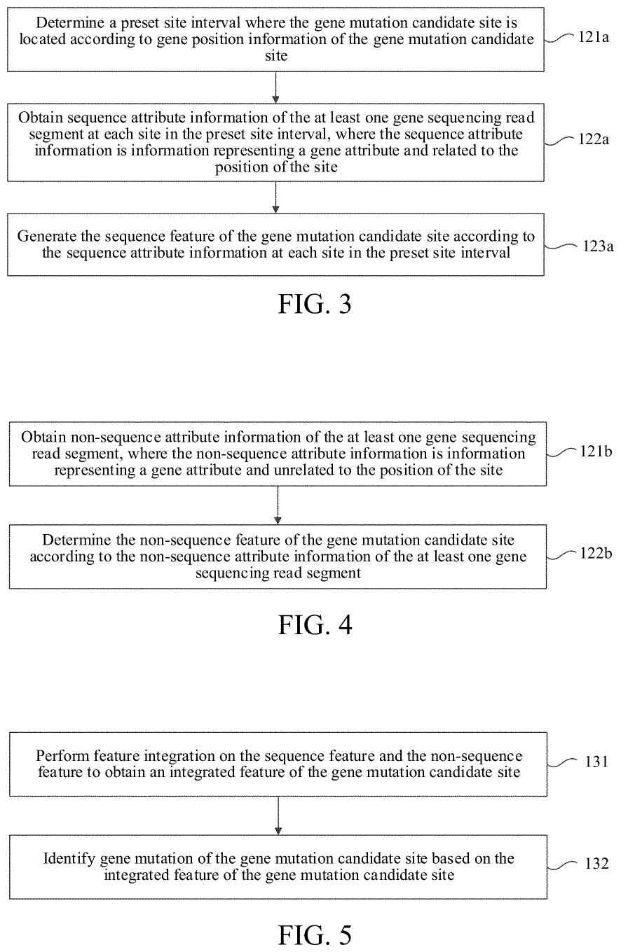

FIG. 3 is a flowchart of a process of determining a sequence feature of a gene mutation candidate site according to the embodiments of the present disclosure. As shown in FIG. 3, step 12 includes the following steps.

At step 121a, a preset site interval where the gene mutation candidate site is located is determined according to gene position information of the gene mutation candidate site.

At step 122a, the sequence attribute information of the at least one gene sequencing read segment at each site in the preset site interval is obtained, where the sequence attribute information is information representing a gene attribute and related to the position of the site.

At step 123a, the sequence feature of the gene mutation candidate site is generated according to the sequence attribute information at each site in the preset site interval.

In an example of the embodiments of the present disclosure, there may be at least one gene sequencing read segment for each gene mutation candidate site. In order to improve the accuracy of gene mutation identification, the sequence attribute information of the gene mutation candidate site can be considered, and the sequence attribute information of the site near the gene mutation candidate site can also be considered. When the sequence feature of the gene mutation candidate site is determined, the preset site interval where the gene mutation candidate site is located can be determined according to the gene position information of the gene mutation candidate site, for example, the interval between 150 base pairs before and after the gene mutation candidate site may serve as the preset site interval where the gene mutation candidate site is located. Then, for each site within the preset site interval, the sequence attribute information of the at least one gene sequencing read segment at the site may be obtained, and the sequence feature corresponding to the site is generated according to the sequence attribute information of the site. The sequence feature may be represented by a sequence feature vector. A sequence feature matrix of the gene mutation candidate site is formed according to at least one sequence feature vector corresponding to at least one site in the preset site interval of the gene mutation candidate site. For example, if the preset site interval of the gene mutation candidate site includes three sites b1, b2, b3, and the sequence feature vectors corresponding to the three sites b1, b2, b3 are al, a2, a3, respectively, the sequence feature matrix of the gene mutation candidate site is [a1, a2, a3], where the sequence features of a1, a2, a3 correspond to the sequence attribute information of b1, b2, b3, respectively.

Here, the sequence attribute information may include, but is not limited to: the gene type of the reference genome; the number of genes of each gene type; the number of deletion genes of each gene type; and the number of insertion genes of each gene type. The gene type of the reference genome may be the gene type of the reference genome at the gene mutation candidate site. The number of genes of each gene type may be the number of genes of each gene type of at least one gene sequencing read segment at the gene mutation candidate site, for example, the gene mutation candidate site corresponds to five gene sequencing read segments, and the gene types of each gene sequencing read segment at the gene mutation candidate site are A, C, C, G, G, respectively, and the number of genes of each gene type is: one A; two Cs; and two Gs. The number of deletion genes of each gene type may be the number of deletion genes of each gene type of at least one gene sequencing read segment at the gene mutation candidate site, for example, the deletion gene types of each gene sequencing read segment at the gene mutation candidate site are A, C, C, G, G, respectively, and the number of deletion genes of each gene type is: one A; two Cs; and two Gs. The number of insertion genes of each gene type may be the number of insertion genes of each gene type of at least one gene sequencing read segment at the gene mutation candidate site, for example, the insertion gene types of each gene sequencing read segment at the gene mutation candidate site are A, C, C, G, G, respectively, and the number of insertion genes of each gene type is: one A; two Cs; and two Gs.

In one possible implementation, when the sequence attribute information of the at least one gene sequencing read segment at each site in the preset site interval is obtained, for each site in the preset site interval, the gene type of the at least one gene sequencing read segment at the site is determined, and the number of genes of each gene type corresponding to the site is counted, so that the at least one gene sequencing read segment corresponding to the gene mutation candidate site is determined, and the number of genes of each gene type at the site is determined.

In one possible implementation, when the sequence attribute information of the at least one gene sequencing read segment at each site in the preset site interval is obtained, for each site in the preset site interval, a gene type of a deletion gene of each gene sequencing read segment at the site is determined according to a comparison result between a gene sequence of each gene sequencing read segment and a gene sequence of a reference genome, and the number of deletion genes of each gene type of the at least one gene sequencing read segment at the site is counted, so that the at least one gene sequencing read segment corresponding to the gene mutation candidate site is determined, and the number of deletion genes of each gene type at the site is determined.

In one possible implementation, when the sequence attribute information of the at least one gene sequencing read segment at each site in the preset site interval is obtained, for each site in the preset site interval, a gene type of an insertion gene of each gene sequencing read segment at the site is determined according to the comparison result between the gene sequence of each gene sequencing read segment and the gene sequence of the reference genome, and the number of insertion genes of each gene type of the at least one gene sequencing read segment at the site is counted, so that the at least one gene sequencing read segment corresponding to the gene mutation candidate site is determined, and the number of insertion genes of each gene type at the site is determined.

For example, assuming that the sequence attribute information includes the gene type of the reference genome, the number of genes of each gene type, the number of deletion genes of each gene type, and the number of insertion genes of each gene type, when the sequence feature of the gene mutation candidate site is determined, for each site in the preset site interval where the gene mutation candidate site are located, four pieces of information of the at least one gene sequencing read segment corresponding to the gene mutation candidate site at the site may be extracted, for example, the gene mutation candidate site corresponds to five gene sequencing read segments, and for a certain site in the preset site interval, the gene type of the reference genome at the site, the number of genes of each gene type of the five gene sequencing read segments at the site, the number of deletion genes of each gene type of the five gene sequencing read segments at this site, and the number of insertion genes of each gene type of the five gene sequencing read segments at this site can be determined respectively. Then, the sequence feature of the site is obtained in combination with at least one piece of sequence attribute information corresponding to the site. The sequence feature of the gene mutation candidate site includes the sequence feature of each site in the preset site interval.

In the example of the embodiments of the present disclosure, when the gene mutation of the gene mutation candidate site is identified, the sequence attribute of the at least one gene sequencing read segment corresponding to the gene mutation candidate site is considered, and the non-sequence attribute of the at least one gene sequencing read segment is also considered. The process of determining the non-sequence feature of the gene mutation candidate site is described in detail below by an example.

FIG. 4 is a flowchart of a process of determining a non-sequence feature of a gene mutation candidate site according to the embodiments of the present disclosure. As shown in FIG. 4, step 12 includes the following steps.

At step 121b, the non-sequence attribute information of the at least one gene sequencing read segment is obtained, where the non-sequence attribute information is information representing a gene attribute and unrelated to the position of the site.

At step 122b, the non-sequence feature of the gene mutation candidate site is generated according to the non-sequence attribute information of the at least one gene sequencing read segment.

In an example of the embodiments of the present disclosure, in order to improve the accuracy of gene mutation identification, sequence attribute information of at least one gene sequencing read segment is considered, and non-sequence attribute information of the at least one gene sequencing read segment is also considered. Here, the non-sequence information includes at least one of the following: comparison quality; positive and negative strand preference; gene sequencing read segment length; or edge preference. When the non-sequence feature of the gene mutation candidate site is determined, the non-sequence attribute information of the at least one gene attribute sequence read segment is obtained, and then the non-sequence feature of the gene mutation candidate site is generated according to the obtained non-sequence attribute information.

In one possible implementation, when the non-sequence feature of the gene mutation candidate site is determined according to the non-sequence attribute information of the at least one gene sequencing read segment, the comparison quality of each gene sequencing read segment is determined according to the comparison quality of each site in each gene sequencing read segment, and then, the non-sequence feature corresponding to the gene mutation candidate site is determined according to the comparison quality of each gene sequencing read segment. Here, the comparison quality can be used for representing the accuracy of gene sequencing of each gene sequence in the gene sequencing read segment. If the comparison quality of a certain gene sequence is less than a preset value, it can be considered that the gene type of the gene sequence obtained through gene sequencing is inaccurate, so that the comparison quality can be used as a reference factor for determining whether the gene of the gene mutation candidate site is mutated. For example, if the gene mutation candidate site corresponds to at least one gene sequencing read segment, the comparison quality of each gene sequencing read segment can be determined according to the comparison quality of each gene sequence. By taking one gene sequencing read segment as an example, an average value or intermediate value of the comparison quality of the gene sequence included in the gene sequencing read segment may serve as the comparison quality of the gene sequencing read segment, and at least one gene sequence can also be randomly selected from the gene sequencing read segment, and the average value or intermediate value of the comparison quality of the at least one selected gene sequence serves as the comparison quality of the gene sequencing read segment. Then, the comparison quality of the gene mutation candidate site is obtained according to the comparison quality of each gene sequencing read segment, for example, an average value or mean value of at least one gene sequencing read segment corresponding to the gene mutation candidate site is calculated to obtain the comparison quality of the gene mutation candidate site, so that the non-sequence feature of the gene mutation candidate site is determined according to the comparison quality of the gene mutation candidate site.

In one possible implementation, when the non-sequence feature of the gene mutation candidate site is determined according to the non-sequence attribute information of the at least one gene sequencing read segment, a positive and negative strand ratio of gene strands to which the at least one gene sequencing read segment belongs is determined according to positive and negative strand information of a gene strand to which each gene sequencing read segment belongs, and then, the non-sequence feature of the gene mutation candidate site is determined according to the determined positive and negative strand ratio. Here, the positive and negative strand preference may be the ratio of the positive and negative strands in the gene strand to which the gene sequencing read segment belongs. The gene strand includes a positive strand and a negative strand, where the positive strand may be a Deoxyribonucleic Acid (DNA) single strand that is the same as a base sequence of Ribonucleic Acid (RNA), and the negative strand may be a DNA single strand that is complementary to the base sequence of RNA. For example, the gene mutation candidate site corresponds to five gene sequencing read segments, where three gene sequencing read segments correspond to the positive strands of the gene strand, two gene sequencing read segments correspond to the negative strands of the gene strand, and then the positive and negative strand preference can be 3:2.

In one possible implementation, when the non-sequence feature of the gene mutation candidate site is determined according to the non-sequence attribute information of the at least one gene sequencing read segment, the non-sequence feature of the gene mutation candidate site is determined according to the gene sequencing read segment length of each gene sequencing read segment. The length of the gene sequencing read segment is the length of the base sequence of each gene sequencing read segment, for example, if one gene sequencing read segment includes four base sequences, the length of the gene sequencing read segment is four. The non-sequence feature of the gene mutation candidate site is determined according to the length of each gene sequencing read segment, and the non-sequence feature of the gene mutation candidate site is also determined according to the intermediate value or average value of the length of the at least one gene sequencing read segment.

In one possible implementation, when the non-sequence feature of the gene mutation candidate site is determined according to the non-sequence attribute information of the at least one gene sequencing read segment, the non-sequence feature of the gene mutation candidate site can be determined according to the edge preference of each gene sequencing read segment. Here, the edge preference may be the ratio of a certain site at an edge position to at an intermediate position of the gene sequencing read segment. For example, the gene sequencing read segment can be averagely divided into three segments, where two segments at the two ends of the gene sequencing read segment may serve as edge positions, and the segment in the middle of the gene sequencing read segment may serve as an intermediate position. The gene mutation candidate site corresponds to five gene sequencing read segments. If the gene mutation candidate site is located at the edge positions of three gene sequencing read segments and located in the middle positions of two gene sequencing read segments, the edge preference of the gene mutation candidate site may be 3:2. Accordingly, the non-sequence feature of the gene mutation candidate site is determined according to the edge preference of the gene mutation candidate site at each gene sequencing read segment, and the non-sequence feature of the gene mutation candidate site is also determined according to the intermediate value or average value of the edge preference corresponding to the at least one gene sequencing read segment.

In this way, the non-sequence feature of the gene mutation candidate site is generated for the non-sequence attribute information of the at least one gene sequencing read segment at the gene mutation candidate site, so that when the gene mutation is identified, the non-sequence feature dimension feature of the gene mutation candidate site is considered, thereby enabling the gene mutation identification to be more accurate. When the non-sequence feature is determined, the non-sequence feature of the at least one gene sequencing read segment is generated according to a combination of any at least one piece of information of the non-sequence attribute information.

The process of identifying gene mutation of a gene mutation candidate site is described below by an example.

FIG. 5 is a flowchart of a process of identifying gene mutation of a gene mutation candidate site according to the embodiments of the present disclosure. As shown in FIG. 5, step 13 includes the following steps.

At step 131, feature integration is performed on the sequence feature and the non-sequence feature to obtain an integrated feature of the gene mutation candidate site.

At step 132, gene mutation of the gene mutation candidate site is identified based on the integrated feature of the gene mutation candidate site.

In the embodiments of the present disclosure, after the sequence feature and the non-sequence dimension feature of the gene mutation candidate site are determined, feature integration of the sequence feature and the non-sequence feature can be performed by using the neural network model, and a sequence feature matrix formed by the sequence feature and a non-sequence feature matrix formed by the non-sequence feature are combined into a feature matrix, so as to obtain an integrated feature matrix formed by feature integration, and then, the gene mutation of the mutation candidate site is identified by using the neural network model according to the integrated feature matrix. In this way, the sequence attribute information and the non-sequence attribute information corresponding to the gene mutation candidate site are integrated by using the neural network model, so that the gene sequencing data is analyzed more comprehensively, and gene mutation identification is more accurate. In the training process, a gene sequencing read segment with Single Nucleotide Polymorphism (SNP) and a gene sequencing read segment with Insertion/Deletion (InDel) may be selected as training samples, so that a trained gene mutation identification model may effectively identify the gene mutation of SNP and InDel.

In one possible implementation, identifying the gene mutation of the gene mutation candidate site based on the integrated feature of the gene mutation candidate site includes: obtaining a mutation value of gene mutation of the gene mutation candidate site according to the integrated feature of the gene mutation candidate site; and if the mutation value is greater than or equal to a preset threshold, determining the existence of gene mutation of the gene mutation candidate site. Here, the mutation value of the gene mutation may be the possibility of representing the mutation of the gene mutation candidate site, for example, the greater the mutation value, the greater the possibility that the gene mutation candidate site is mutated. A two-dimensional feature is processed by using the neural network to obtain a mutation value, and whether gene mutation of the gene mutation candidate site exists is determined according to the mutation value. In one possible implementation, the mutation value may be between 0 and 1. A preset threshold may be set according to an application scene, e.g., 0.3 and 0.5, if the mutation value is greater than the preset threshold, it can be considered that the gene of the gene mutation candidate site is mutated, and otherwise, the gene of the gene mutation candidate site is not mutated.

In the embodiments of the present disclosure, the gene mutation of the gene mutation candidate site is identified by using the neural network model, and the neural network model may extract the sequence feature and the non-sequence feature of the gene mutation candidate site. The embodiments of the present disclosure further provide a structure of the neural network model.

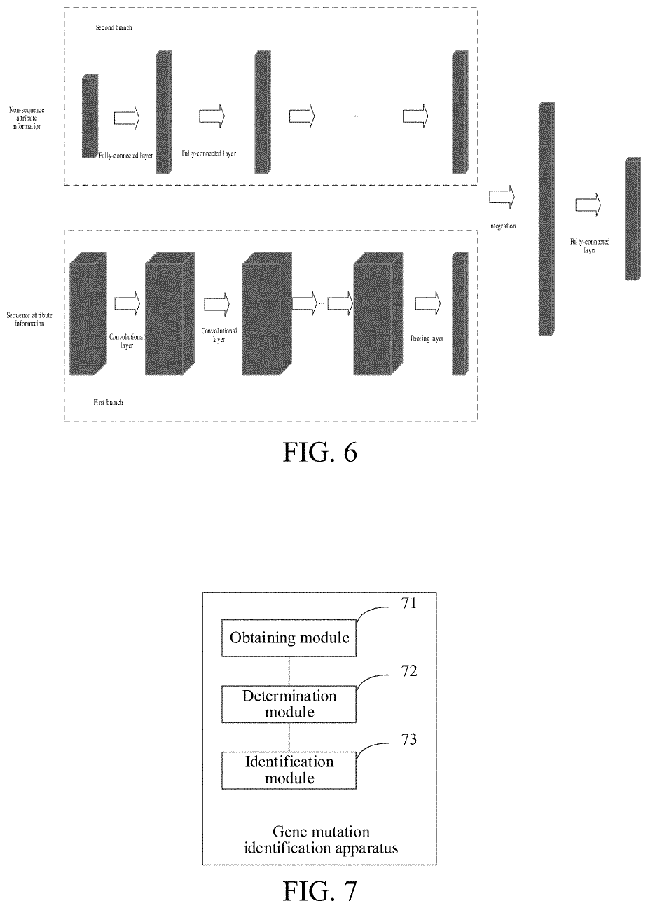

FIG. 6 is a block diagram of a neural network model according to the embodiments of the present disclosure. As shown in FIG. 6, the neural network model includes two branch structures, i.e., a first branch and a second branch. The first branch may be used for extracting the sequence feature of the at least one gene sequencing read segment corresponding to the gene mutation candidate site, and the first branch may include a convolutional layer and a pooling layer. The second branch may be used for extracting the non-sequence feature of the at least one gene sequencing read segment corresponding to the gene mutation candidate site, and the second branch structure may include a fully-connected layer. After the neural network model extracts the sequence feature and the non-sequence feature of the gene mutation candidate site, the sequence feature and the non-sequence feature are integrated, for example, the sequence feature matrix of the sequence feature and the non-sequence feature matrix of the non-sequence feature are spliced to obtain an integrated feature matrix of an integrated feature, and then, the mutation value of the gene mutation candidate site is obtained through the fully-connected layer.

In the embodiments of the present disclosure, sequence attribute information and non-sequence attribute information of at least one gene sequencing read segment corresponding to a gene mutation candidate site are extracted, and gene mutation is identified using an integrated feature that integrates the sequence attribute information and the non-sequence attribute information, so that the sequence attribute information and the non-sequence attribute information corresponding to the gene mutation candidate site are comprehensively considered, thereby more comprehensively analyzing gene sequencing information, better identifying gene mutation of the gene mutation candidate site, screening out germ line gene mutation and avoiding interference caused by noise and errors, and improving the accuracy of gene mutation identification.

A person skilled in the art can understand that, in the foregoing methods of the specific implementations, the order in which the steps are written does not imply a strict execution order which constitutes any limitation to the implementation process, and the specific order of executing the steps should be determined by functions and possible internal logics thereof

FIG. 7 is a block diagram of a gene mutation identification apparatus according to the embodiments of the present disclosure. As shown in FIG. 7, the gene mutation identification apparatus includes:
an obtaining module 71, configured to obtain at least one gene sequencing read segment corresponding to a gene mutation candidate site;
a determination module 72, configured to determine a sequence feature and a non-sequence feature of the gene mutation candidate site according to attribute information of the at least one gene sequencing read segment, where the sequence feature is a feature related to the position of the site; and
an identification module 73, configured to identify gene mutation of the gene mutation candidate site based on the sequence feature and the non-sequence feature.

In one possible implementation, the attribute information includes sequence attribute information; and the determination module 72 includes:
a first determination sub-module, configured to determine a preset site interval where the gene mutation candidate site is located according to gene position information of the gene mutation candidate site;
a first obtaining sub-module, configured to obtain the sequence attribute information of the at least one gene sequencing read segment at each site in the preset site interval, where the sequence attribute information is information representing a gene attribute and related to the position of the site; and
a first generation sub-module, configured to generate the sequence feature of the gene mutation candidate site according to the sequence attribute information at each site in the preset site interval.

In one possible implementation, the first obtaining sub-module is specifically configured to: determine a gene type of the at least one gene sequencing read segment at each site; and count the number of genes of each gene type corresponding to each site.

In one possible implementation, the first obtaining sub-module is specifically configured to: determine a gene type of a deletion gene of each gene sequencing read segment at each site according to a comparison result between a gene sequence of each gene sequencing read segment and a gene sequence of a reference genome; and count the number of deletion genes of each gene type of the at least one gene sequencing read segment at each site.

In one possible implementation, the first obtaining sub-module is specifically configured to: determine a gene type of an insertion gene of each gene sequencing read segment at each site according to the comparison result between the gene sequence of each gene sequencing read segment and the gene sequence of the reference genome; and count the number of insertion genes of each gene type of the at least one gene sequencing read segment at each site.

In one possible implementation, the sequence attribute information includes at least one of the following:
the gene type of a reference gene; the number of genes of each gene type; the number of deletion genes of each gene type; or the number of insertion genes of each gene type.

In one possible implementation, the attribute information includes non-sequence attribute information; and the determination module includes:
a second obtaining sub-module, configured to obtain the non-sequence attribute information of the at least one gene sequencing read segment, where the non-sequence attribute information is information representing a gene attribute and unrelated to the position of the site; and
a second determination sub-module, configured to determine the non-sequence feature of the gene mutation candidate site according to the non-sequence attribute information of the at least one gene sequencing read segment.

In one possible implementation, the non-sequence information includes at least one of the following:
comparison quality; positive and negative strand preference; gene sequencing read segment length; or edge preference.

In one possible implementation, the second determination sub-module is specifically configured to: determine the comparison quality of each gene sequencing read segment according to the comparison quality of each site in each gene sequencing read segment, where the comparison quality is used for representing the accuracy of gene sequencing of each gene sequence in the gene sequencing read segment; and determine the non-sequence feature corresponding to the gene mutation candidate site according to the comparison quality of each gene sequencing read segment.

In one possible implementation, the second determination sub-module is specifically configured to: determine a positive and negative strand ratio of gene strands to which the at least one gene sequencing read segment belongs according to positive and negative strand information of a gene strand to which each gene sequencing read segment belongs; and determine the non-sequence feature corresponding to the gene mutation candidate site according to the positive and negative strand ratio.

In one possible implementation, the identification module 73 includes:
an integration sub-module, specifically configured to perform feature integration on the sequence feature and the non-sequence feature to obtain an integrated feature of the gene mutation candidate site; and
an identification sub-module, configured to identify gene mutation of the gene mutation candidate site based on the integrated feature of the gene mutation candidate site.

In one possible implementation, the identification sub-module is specifically configured to: obtain a mutation value of gene mutation of the gene mutation candidate site according to the integrated feature of the gene mutation candidate site; and if the mutation value is greater than or equal to a preset threshold, determine the existence of gene mutation of the gene mutation candidate site.

In one possible implementation, the obtaining module 71 is specifically configured to:
obtain a gene sequencing read segment obtained by performing gene sequencing on a somatic gene;
compare the gene sequence of the gene sequencing read segment with the gene sequence of the reference genome to obtain a comparison result;
determine the gene mutation candidate site of an abnormal gene of the somatic gene according to the comparison result; and
obtain at least one gene sequencing read segment corresponding to the gene mutation candidate site.

In some embodiments, functions or modules included in the apparatus provided in the embodiments of the present disclosure may be configured to perform the method described in the foregoing method embodiments. For specific implementation of the apparatus, reference may be made to descriptions of the foregoing method embodiments. For brevity, details are not described here again.

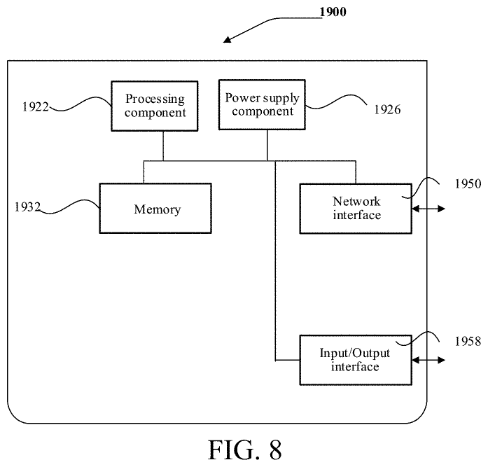

FIG. 8 is a block diagram of a gene mutation identification apparatus 1900 illustrated according to an exemplary embodiment. For example, the apparatus 1900 may be provided as a server. Referring to FIG. 8, the apparatus 1900 includes a processing component 1922 that further includes one or more processors; and a memory resource represented by a memory 1932, configured to store instructions, for example, an application program, that may be executed by the processing component 1922. The application program stored in the memory 1932 may include one or more modules each corresponding to a set of instructions. In addition, the processing component 1922 is configured to execute the instructions to perform the foregoing method.

The apparatus 1900 may further include: a power supply component 1926, configured to perform power management of the apparatus 1900; a wired or wireless network interface 1950, configured to connect the apparatus 1900 to a network; and an Input/Output (I/O) interface 1958. The apparatus 1900 may operate an operating system stored in the memory 1932, such as Windows Server™, Mac OS X™, Unix™, Linux™, or FreeBSD™.

In an exemplary embodiment, a non-volatile computer-readable storage medium, for example, the memory 1932 including computer program instructions, is further provided. The computer program instructions may be executed by the processing component 1922 in the apparatus 1900 to complete the foregoing method.

The present disclosure may be a system, a method, and/or a computer program product. The computer program product may include a computer-readable storage medium, and computer-readable program instructions that are used by a processor to implement various aspects of the present disclosure are loaded on the computer-readable storage medium.

The computer-readable storage medium may be a tangible device that can maintain and store instructions used by an instruction execution device. The computer-readable storage medium may be, but is not limited to, an electronic storage device, a magnetic storage device, an optical storage device, an electromagnetic storage device, a semiconductor storage device, or any suitable combination of the above ones. More specific examples (a non-exhaustive list) of the computer-readable storage medium include a portable computer disk, a hard disk, a Random Access Memory (RAM), a Read-Only Memory (ROM), an Erasable Programmable Read-Only Memory (EPROM or Flash memory), a Static Random Access Memory (SRAM), a portable Compact Disk Read-Only Memory (CD-ROM), a Digital Versatile Disc (DVD), a memory stick, a floppy disk, a mechanically encoded device such as punch-cards or raised structure in a groove having instructions stored thereon, and any suitable combination of the foregoing. The computer-readable storage medium used here is not interpreted as an instantaneous signal such as a radio wave or another freely propagated electromagnetic wave, an electromagnetic wave propagated by a waveguide or another transmission medium (for example, an optical pulse transmitted by an optical fiber cable), or an electrical signal transmitted by a wire.

The computer-readable program instructions described here may be downloaded from a computer-readable storage medium to each computing/processing device, or downloaded to an external computer or an external storage device via a network, such as the Internet, a Local Area Network (LAN), a Wide Area Network (WAN), and/or a wireless network. The network may include a copper transmission cable, optical fiber transmission, wireless transmission, a router, a firewall, a switch, a gateway computer, and/or an edge server. A network adapter or a network interface in each computing/processing device receives the computer-readable program instructions from the network, and forwards the computer-readable program instructions, so that the computer-readable program instructions are stored in a computer-readable storage medium in each computing/processing device.

Computer program instructions for carrying out operations of the present disclosure may be assembler instructions, Instruction-Set-Architecture (ISA) instructions, machine instructions, machine dependent instructions, microcode, firmware instructions, state-setting data, or either source code or target code written in any combination of one or more programming languages, including an object-oriented programming language such as Smalltalk or C++, and conventional procedural programming languages, such as the “C” programming language or similar programming languages. The computer-readable program instructions may be completely executed on a user computer, partially executed on a user computer, executed as an independent software package, executed partially on a user computer and partially on a remote computer, or completely executed on a remote computer or a server. In the case of a remote computer, the remote computer may be connected to a user computer via any kind of network, including an LAN or a WAN, or may be connected to an external computer (for example, connected via the Internet with the aid of an Internet service provider). In some embodiments, an electronic circuit such as a programmable logic circuit, a Field-Programmable Gate Array (FPGA), or a Programmable Logic Array (PLA) is personalized by using status information of the computer-readable program instructions, and the electronic circuit may execute the computer-readable program instructions to implement various aspects of the present disclosure.

The aspects of the present disclosure are described herein with reference to flowcharts and/or block diagrams of methods, apparatuses (systems), and computer program products according to the embodiments of the present disclosure. It should be understood that each block in the flowcharts and/or block diagrams and a combination of the blocks in the flowcharts and/or block diagrams may be implemented by using the computer-readable program instructions.

These computer-readable program instructions may be provided to a processor of a general-purpose computer, a special-purpose computer, or other programmable data processing apparatus to produce a machine, such that the instructions, when executed via the processor of the computer or other programmable data processing apparatus, generate an apparatus for implementing a specified function/action in one or more blocks in the flowcharts and/or block diagrams. These computer-readable program instructions may also be stored in a computer-readable storage medium, and these instructions may instruct a computer, a programmable data processing apparatus, and/or another device to work in a specific manner. Therefore, the computer-readable storage medium storing the instructions includes an artifact, and the artifact includes instructions for implementing the aspects of a specified function/action in one or more blocks in the flowcharts and/or block diagrams.

The computer-readable program instructions may be loaded onto a computer, another programmable data processing apparatus, or another device, so that a series of operations and steps are executed on the computer, the another programmable apparatus, or the another device, thereby generating computer-implemented processes. Therefore, the instructions executed on the computer, the another programmable apparatus, or the another device implement a specified function/action in one or more blocks in the flowcharts and/or block diagrams.

The flowcharts and block diagrams in the accompanying drawings illustrate the architecture, functionality and operations of possible implementations of systems, methods, and computer program products according to the embodiments of the present disclosure. In this regard, each block in the flowcharts or block diagrams may represent a module, a program segment, or a part of instruction, and the module, the program segment, or the part of instruction includes one or more executable instructions for implementing a specified logical function. In some alternative implementations, functions marked in the block may also occur in an order different from that marked in the accompanying drawings. For example, two consecutive blocks are actually executed substantially in parallel, or are sometimes executed in a reverse order, depending on the involved functions. It should also be noted that each block in the block diagrams and/or flowcharts and a combination of blocks in the block diagrams and/or flowcharts may be implemented by using a dedicated hardware-based system that executes a specified function or action, or may be implemented by using a combination of dedicated hardware and a computer instruction.

The embodiments of the present disclosure are described above. The foregoing descriptions are exemplary but not exhaustive, and are not limited to the disclosed embodiments. For a person of ordinary skill in the art, many modifications and mutations are all obvious without departing from the scope and spirit of the described embodiments. The terms used herein are intended to best explain the principles of the embodiments, practical applications, or technical improvements to the technologies in the market, or to enable other persons of ordinary skill in the art to understand the embodiments disclosed herein.

1. A gene mutation identification method, comprising:
obtaining at least one gene sequencing read segment corresponding to a gene mutation candidate site;
determining a sequence feature and a non-sequence feature of the gene mutation candidate site according to attribute information of the at least one gene sequencing read segment, wherein the sequence feature is a feature related to the position of the site; and
identifying gene mutation of the gene mutation candidate site based on the sequence feature and the non-sequence feature.

2. The method according to claim 1, wherein the attribute information comprises sequence attribute information; and determining the sequence feature of the gene mutation candidate site according to the attribute information of the at least one gene sequencing read segment comprises:
determining a preset site interval where the gene mutation candidate site is located according to gene position information of the gene mutation candidate site;
obtaining the sequence attribute information of the at least one gene sequencing read segment at each site in the preset site interval, wherein the sequence attribute information is information representing a gene attribute and related to the position of the site; and
generating the sequence feature of the gene mutation candidate site according to the sequence attribute information at each site in the preset site interval.

3. The method according to claim 2,
wherein obtaining the sequence attribute information of the at least one gene sequencing read segment at each site in the preset site interval comprises:
determining a gene type of the at least one gene sequencing read segment at each site; and
counting the number of genes of each gene type corresponding to each site, or
wherein obtaining the sequence attribute information of the at least one gene sequencing read segment at each site in the preset site interval comprises:
determining a gene type of a deletion gene of each gene sequencing read segment at each site according to a comparison result between a gene sequence of each gene sequencing read segment and a gene sequence of a reference genome; and
counting the number of deletion genes of each gene type of the at least one gene sequencing read segment at each site, or
wherein obtaining the sequence attribute information of the at least one gene sequencing read segment at each site in the preset site interval comprises:
determining a gene type of an insertion gene of each gene sequencing read segment at each site according to the comparison result between the gene sequence of each gene sequencing read segment and the gene sequence of the reference genome; and
counting the number of insertion genes of each gene type of the at least one gene sequencing read segment at each site.

4. The method according to claim 1, wherein the sequence attribute information comprises at least one of the following:
the gene type of a reference gene; the number of genes of each gene type; the number of deletion genes of each gene type; or the number of insertion genes of each gene type.

5. The method according to claim 1, wherein the attribute information comprises non-sequence attribute information; and determining the non-sequence feature of the gene mutation candidate site according to the attribute information of the at least one gene sequencing read segment comprises:
obtaining the non-sequence attribute information of the at least one gene sequencing read segment, wherein the non-sequence attribute information is information representing a gene attribute and unrelated to the position of the site; and
determining the non-sequence feature of the gene mutation candidate site according to the non-sequence attribute information of the at least one gene sequencing read segment.

6. The method according to claim 5, wherein the non-sequence information comprises at least one of the following:
comparison quality; positive and negative strand preference; gene sequencing read segment length; or edge preference.

7. The method according to claim 6,
wherein determining the non-sequence feature of the gene mutation candidate site according to the non-sequence attribute information of the at least one gene sequencing read segment comprises:
determining the comparison quality of each gene sequencing read segment according to the comparison quality of each site in each gene sequencing read segment, wherein the comparison quality is used for representing the accuracy of gene sequencing of each gene sequence in the gene sequencing read segment; and
determining the non-sequence feature corresponding to the gene mutation candidate site according to the comparison quality of each gene sequencing read segment, or
wherein determining the non-sequence feature of the gene mutation candidate site according to the non-sequence attribute information of the at least one gene sequencing read segment comprises:
determining a positive and negative strand ratio of gene strands to which the at least one gene sequencing read segment belongs according to positive and negative strand information of a gene strand to which each gene sequencing read segment belongs; and
determining the non-sequence feature corresponding to the gene mutation candidate site according to the positive and negative strand ratio.

8. The method according to claim 1, wherein identifying the gene mutation of the gene mutation candidate site based on the sequence feature and the non-sequence feature comprises:
performing feature integration on the sequence feature and the non-sequence feature to obtain an integrated feature of the gene mutation candidate site; and
identifying the gene mutation of the gene mutation candidate site based on the integrated feature of the gene mutation candidate site.

9. The method according to claim 8, wherein identifying the gene mutation of the gene mutation candidate site based on the integrated feature of the gene mutation candidate site comprises:
obtaining a mutation value of gene mutation of the gene mutation candidate site according to the integrated feature of the gene mutation candidate site; and
if the mutation value is greater than or equal to a preset threshold, determining the existence of gene mutation of the gene mutation candidate site.

10. The method according to claim 1, wherein obtaining the at least one gene sequencing read segment corresponding to the gene mutation candidate site comprises:
obtaining a gene sequencing read segment obtained by performing gene sequencing on a somatic gene;
comparing the gene sequence of the gene sequencing read segment with the gene sequence of the reference genome to obtain a comparison result;
determining the gene mutation candidate site of an abnormal gene of the somatic gene according to the comparison result; and
obtaining the at least one gene sequencing read segment corresponding to the gene mutation candidate site.

11. A gene mutation identification apparatus, comprising:
a processor; and
a memory configured to store processor-executable instructions,
wherein the processor is configured to invoke the instructions stored in the memory, so as to:
obtain at least one gene sequencing read segment corresponding to a gene mutation candidate site;
determine a sequence feature and a non-sequence feature of the gene mutation candidate site according to attribute information of the at least one gene sequencing read segment, wherein the sequence feature is a feature related to the position of the site; and
identify gene mutation of the gene mutation candidate site based on the sequence feature and the non-sequence feature.

12. The apparatus according to claim 11, wherein the attribute information comprises sequence attribute information; and determining the sequence feature and the non-sequence feature of the gene mutation candidate site according to the attribute information of the at least one gene sequencing read segment comprises:
determining a preset site interval where the gene mutation candidate site is located according to gene position information of the gene mutation candidate site;
obtaining the sequence attribute information of the at least one gene sequencing read segment at each site in the preset site interval, wherein the sequence attribute information is information representing a gene attribute and related to the position of the site; and
generating the sequence feature of the gene mutation candidate site according to the sequence attribute information at each site in the preset site interval.

13. The apparatus according to claim 12,
wherein obtaining the sequence attribute information of the at least one gene sequencing read segment at each site in the preset site interval comprises:
determining a gene type of the at least one gene sequencing read segment at each site; and
counting the number of genes of each gene type corresponding to each site, or
wherein obtaining the sequence attribute information of the at least one gene sequencing read segment at each site in the preset site interval comprises:
determining a gene type of a deletion gene of each gene sequencing read segment at each site according to a comparison result between a gene sequence of each gene sequencing read segment and a gene sequence of a reference genome; and
counting the number of deletion genes of each gene type of the at least one gene sequencing read segment at each site, or
wherein obtaining the sequence attribute information of the at least one gene sequencing read segment at each site in the preset site interval comprises:
determining a gene type of an insertion gene of each gene sequencing read segment at each site according to the comparison result between the gene sequence of each gene sequencing read segment and the gene sequence of the reference genome; and
counting the number of insertion genes of each gene type of the at least one gene sequencing read segment at each site.

14. The apparatus according to claim 11, wherein the sequence attribute information comprises at least one of the following:
the gene type of a reference gene; the number of genes of each gene type; the number of deletion genes of each gene type; or the number of insertion genes of each gene type.

15. The apparatus according to claim 11, wherein the attribute information comprises non-sequence attribute information; and determining the sequence feature and the non-sequence feature of the gene mutation candidate site according to the attribute information of the at least one gene sequencing read segment comprises:
obtaining the non-sequence attribute information of the at least one gene sequencing read segment, wherein the non-sequence attribute information is information representing a gene attribute and unrelated to the position of the site; and
determining the non-sequence feature of the gene mutation candidate site according to the non-sequence attribute information of the at least one gene sequencing read segment.

16. The apparatus according to claim 15, wherein the non-sequence information comprises at least one of the following:
comparison quality; positive and negative strand preference; gene sequencing read segment length; or edge preference.

17. The apparatus according to claim 16,
wherein determining the non-sequence feature of the gene mutation candidate site according to the non-sequence attribute information of the at least one gene sequencing read segment comprises:
determining the comparison quality of each gene sequencing read segment according to the comparison quality of each site in each gene sequencing read segment, wherein the comparison quality is used for representing the accuracy of gene sequencing of each gene sequence in the gene sequencing read segment; and
determining the non-sequence feature corresponding to the gene mutation candidate site according to the comparison quality of each gene sequencing read segment, or
wherein determining the non-sequence feature of the gene mutation candidate site according to the non-sequence attribute information of the at least one gene sequencing read segment comprises:
determining a positive and negative strand ratio of gene strands to which the at least one gene sequencing read segment belongs according to positive and negative strand information of a gene strand to which each gene sequencing read segment belongs; and
determining the non-sequence feature corresponding to the gene mutation candidate site according to the positive and negative strand ratio.

18. The apparatus according to claim 11, wherein identifying the gene mutation of the gene mutation candidate site based on the sequence feature and the non-sequence feature comprises:
performing feature integration on the sequence feature and the non-sequence feature to obtain an integrated feature of the gene mutation candidate site; and
identifying the gene mutation of the gene mutation candidate site based on the integrated feature of the gene mutation candidate site.

19. The apparatus according to claim 18, wherein identifying the gene mutation of the gene mutation candidate site based on the integrated feature of the gene mutation candidate site comprises: obtaining a mutation value of gene mutation of the gene mutation candidate site according to the integrated feature of the gene mutation candidate site; and if the mutation value is greater than or equal to a preset threshold, determining the existence of gene mutation of the gene mutation candidate site.

20. The apparatus according to claim 11, wherein obtaining the at least one gene sequencing read segment corresponding to the gene mutation candidate site comprises:
obtaining a gene sequencing read segment obtained by performing gene sequencing on a somatic gene;
comparing the gene sequence of the gene sequencing read segment with the gene sequence of the reference genome to obtain a comparison result;
determining the gene mutation candidate site of an abnormal gene of the somatic gene according to the comparison result; and
obtaining the at least one gene sequencing read segment corresponding to the gene mutation candidate site.

21. A non-transitory computer-readable storage medium, having computer program instructions stored thereon, wherein when the computer program instructions are executed by a processor, the processor is caused to perform the operations of:
obtaining at least one gene sequencing read segment corresponding to a gene mutation candidate site;
determining a sequence feature and a non-sequence feature of the gene mutation candidate site according to attribute information of the at least one gene sequencing read segment, wherein the sequence feature is a feature related to the position of the site; and
identifying gene mutation of the gene mutation candidate site based on the sequence feature and the non-sequence feature.
